(19) United States
(12) Patent Application Publication
(10) Pub. No.: US 2011/0112361 A1
(43) Pub. Date: May 12, 2011

(54) ENDOSCOPE DEVICE

(75) Inventors: Takakazu ISHIGAMI, Tokyo (JP); Ayumu INADA, Tokyo (JP)

(73) Assignee: OLYMPUS CORPORATION, Tokyo (JP)

(21) Appl. No.: 12/909,067

(22) Filed: Oct. 21, 2010

(30) Foreign Application Priority Data
Nov. 6, 2009 (JP) P2009-254899

(51) Int. Cl. A61B 1/04

(52) U.S. Cl. 600/109; 600/118

(57) ABSTRACT

An endoscope device has a scope unit that is used to observe an interior of a specimen subject, and a main body unit that controls the scope unit by communicating with the scope unit. The scope unit has an insertion portion, a bending portion that is provided on the insertion portion, a bending drive unit that drives the bending portion, an imaging unit that is provided on the insertion portion and acquires images of the specimen subject; and a structure storage section in which structure identification information that is based on the structure of the first scope unit is stored. The main body unit has a display unit that displays images acquired by the imaging unit, an operating section that is used to make operation inputs to operate at least the bending portion, and a control unit that controls the bending drive unit based on the structure identification information stored in the storage section.

### BACKGROUND OF THE INVENTION

1. Field of the Invention

The present invention relates to an endoscope device that is used to observe internal portions of an examination subject. Priority is claimed on Japanese Patent Application No. 2009-254899, filed Nov. 6, 2009, the contents of which are incorporated herein by reference.

2. Description of Related Art

Conventionally, an endoscope device provided with an elongated insertion portion is widely used in order to observe internal portions of an examination subject. A structure is known for this type of endoscope device in which an imaging device formed by a CCD or the like is provided on a distal end of the insertion portion. In this type of endoscope device, images acquired by the imaging device are transmitted through the insertion portion to a main body of the endoscope device. The images then undergo image processing and are displayed on a display unit of a display monitor or the like.

Moreover, in an endoscope device, a structure in which there is provided a bending mechanism that bends the distal end of the insertion portion in order to aim the imaging device at an observation subject which is an internal portion of an examination subject is common. Moreover, because, in many cases, light does not reach as far as the internal portions of an examination subject, a technique in which the visual field of the imaging device is made brighter (i.e., is illuminated) using an illumination mechanism that includes a light emitting component such as a light emitting diode (LED) or the like is practiced in conventional endoscope devices.

In recent years, in order to make endoscopes easier to use, devices have been proposed (see, for example, United States Patent Application Publication No. 2009/0109429) in which an operation section that operates the insertion portion and the above described display unit are integrated into a single unit, and that are sufficiently miniaturized that they can be held in one hand. In this type of endoscope device, the operating section, display unit, and main body unit are collected in a single housing, and an examiner uses the endoscope by directly holding the housing.

### SUMMARY OF THE INVENTION

The present invention proposes the following devices.

The endoscope device of the present invention is provided with a first scope unit that is used to observe a specimen subject, and with a main body unit that can be removably attached to the first scope unit, and that, when the first scope unit is loaded in position, controls the first scope unit by communicating with the first scope unit. In addition, the first scope unit has an insertion portion, a bending portion that is provided on the insertion portion, a bending drive unit that drives the bending portion, an observation device that is provided on the insertion portion and acquires images of the specimen subject, and a storage section in which structure identification information that is based on the structure of the first scope unit is stored. In addition, the main body unit has a display unit that displays images acquired by the observation device, an operating section that is used to make operation inputs to operate at least the bending portion, and a control unit that controls the bending drive unit based on the structure identification information stored in the storage section.

### BRIEF DESCRIPTION OF THE DRAWINGS

FIG. 1 is a perspective view showing an endoscope device according to an embodiment of the present invention.

FIG. 2 is a system structure diagram showing the structure of an endoscope system according to an embodiment of the present invention.

FIG. 3A is a front view showing a state in which a scope unit is mounted on this endoscope system.

FIG. 3B is a partial cross-sectional view showing a state in which a scope unit is mounted on this endoscope system.

FIG. 4 is a partial cross-sectional view showing a state in which another scope unit is mounted on this endoscope system.

FIG. 5 is a block diagram showing the structure of this endoscope system.

FIG. 6 is a flowchart showing an operation when this endoscope system is in use.

FIG. 7 is a flowchart showing an operation when this endoscope system is in use.

FIG. 8 is a flowchart showing an operation when this endoscope system is in use.

FIGS. 9A and 9B are side views showing the structure of a scope unit in variant example 1 of this endoscope system.

FIGS. 10A and 10B are side views showing the structure of a scope unit in variant example 2 of this endoscope system.

FIG. 11 is a side view showing the structure of a scope unit in variant example 3 of this endoscope system.

FIG. 12 is a perspective view showing the structure of a scope unit in variant example 4 of this endoscope system.

FIG. 13 is a partial cross-sectional view showing an endoscope device of variant example 4 on which the scope unit has been mounted.

FIG. 14 is a block diagram showing the structure of variant example 5 of this endoscope system.

FIG. 15 is a block diagram showing another example of the structure of variant example 5 of this endoscope system.

FIGS. 16A through 16C are perspective views showing the structure of a scope unit of variant example 6 of this endoscope system.

### DETAILED DESCRIPTION OF THE INVENTION

Hereinafter, an endoscope system of an embodiment of the present invention will be described.

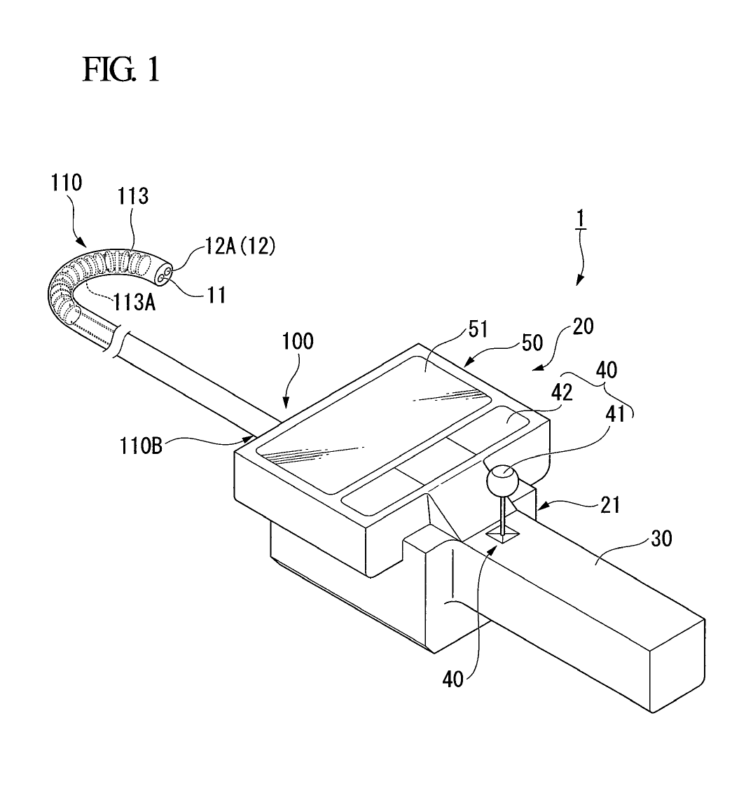

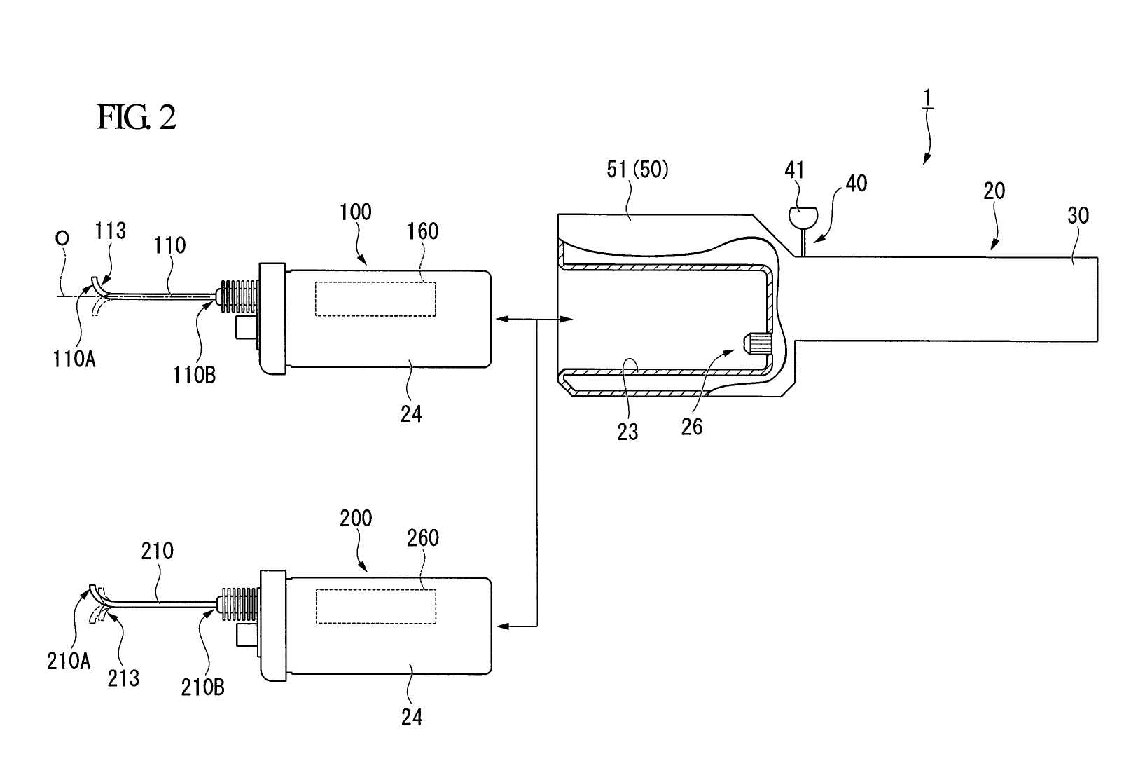

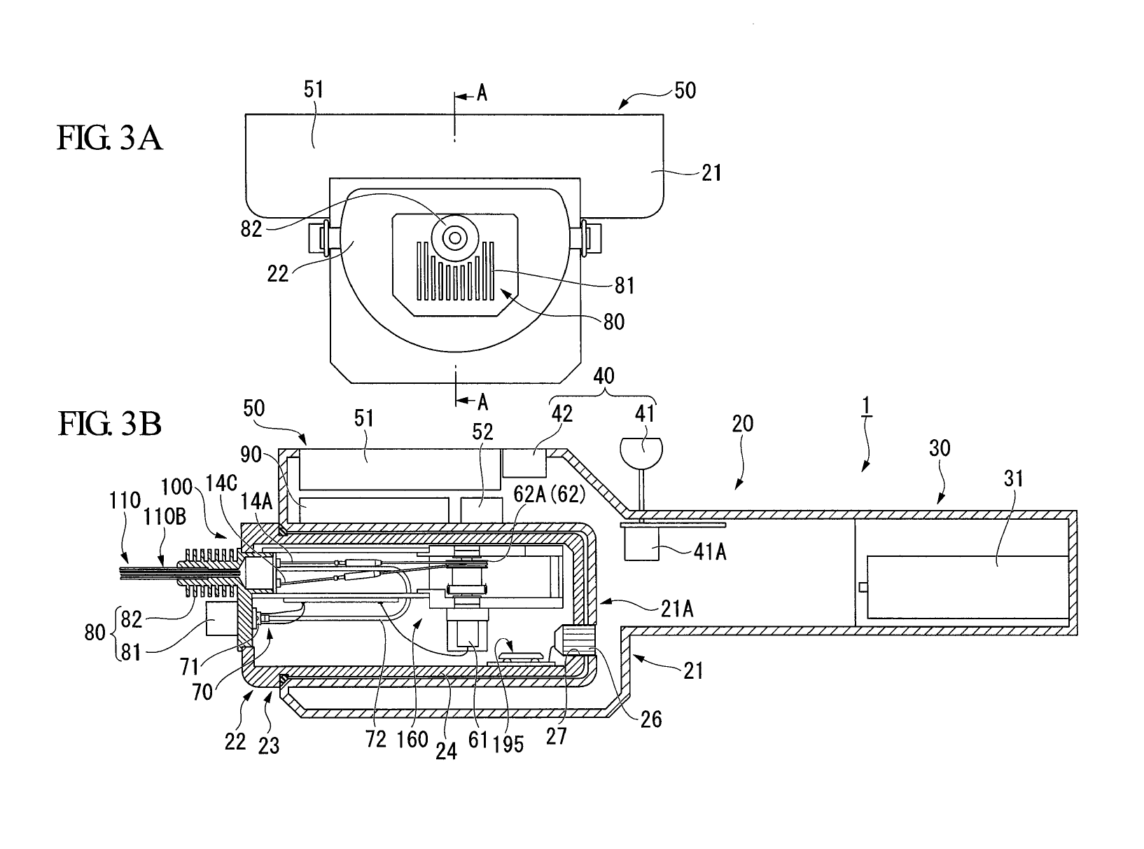

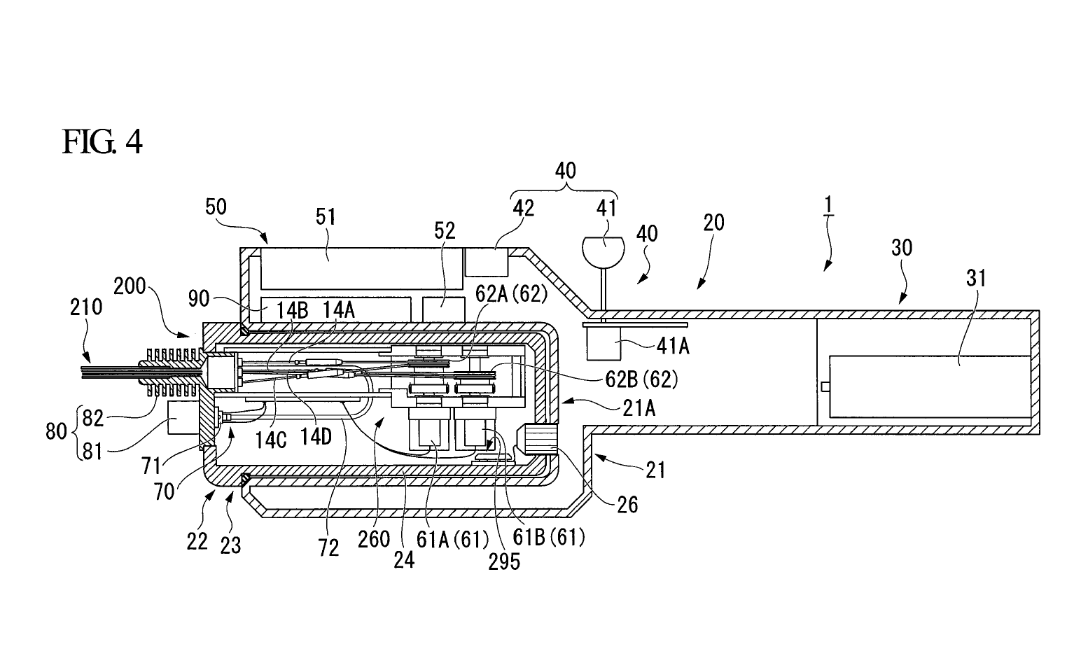

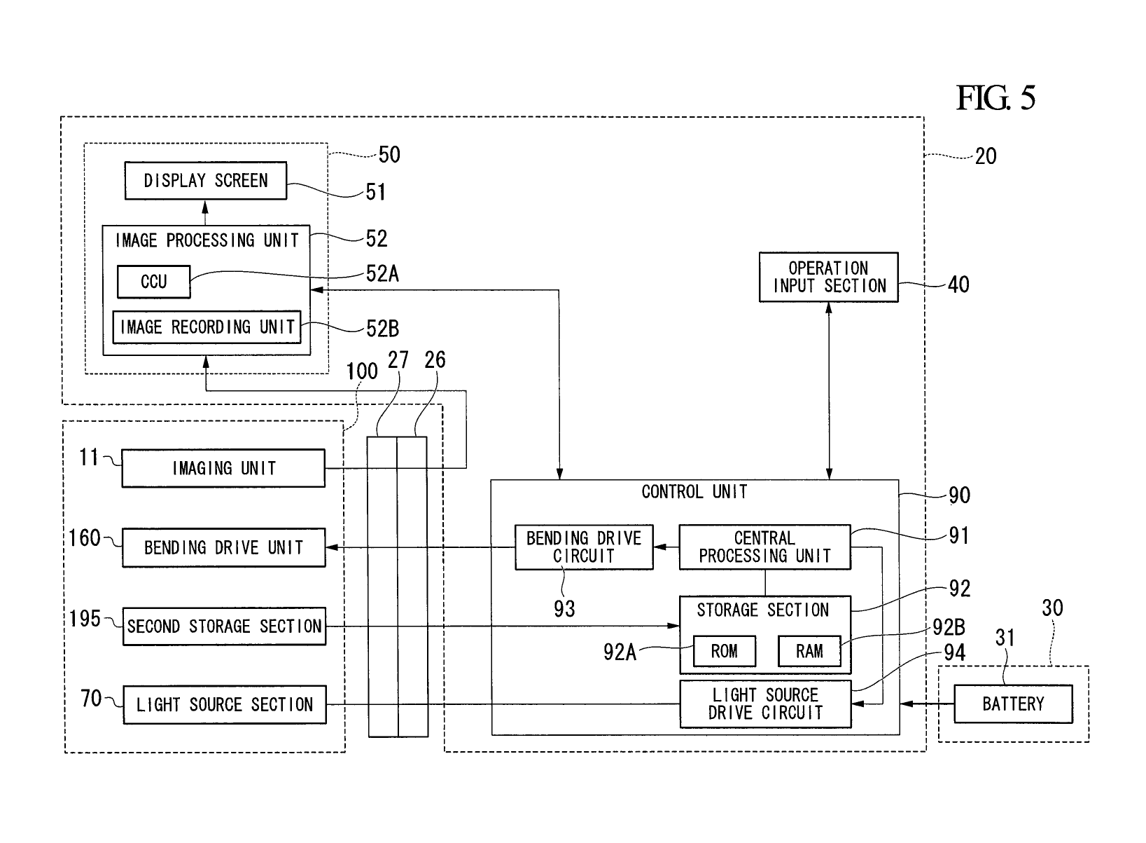

Firstly, the structure of the endoscope system of the present embodiment will be described with reference made to FIGS. 1 through 5. FIG. 1 is a perspective view showing an endoscope system 1 of the present embodiment. FIG. 2 is a partial cross-sectional view showing the endoscope system 1. FIG. 3 shows a state in which a scope unit 100 is mounted on the endoscope system 1, with FIG. 3A being a front view and FIG. 3B a side cross-sectional view thereof. FIG. 4 is a side cross-sectional view showing a state in which a scope unit 200 is mounted on the endoscope system 1. FIG. 5 is a block diagram showing the structure of the endoscope system 1.

As is shown in FIG. 1 and FIG. 2, the endoscope system 1 is provided with a scope unit 100 having an insertion portion 110, and a main body unit 20 that is connected to a proximal end side of the insertion portion 110. The scope unit 100 is a component that is used to observe internal portions of an examination subject. The endoscope system 1 is further provided with a scope unit 200 which is able to be switched with the scope unit 100. The endoscope system 1 of the present embodiment operates as an endoscope device both when the scope unit 100 is mounted on the main body unit 20, and when the scope unit 200 is mounted on the main body unit 20.

The insertion portion 110 that is provided on the scope unit 100 is a component that is inserted into an internal portion of an examination subject, and is formed as an elongated tube-shaped component that has flexibility. The insertion portion 110 is provided with an imaging unit 11 and an illumination unit 12 that are provided at a distal end 110A of the insertion portion 110, and with a bending portion 113 that is used to change the orientation of the distal end of the insertion portion 110 to a desired direction.

The imaging unit 11 is provided with an objective optical system (not shown) that forms images of an observation location in the internal portion of the examination subject, and with an imaging element such as a CCD or the like that forms images by acquiring the images of the observation location formed by the objective optical system and then converting these into an image signal by photoelectric conversion. A signal wire (not shown) that is used for transmitting the image signals of the images acquired by the imaging element is connected to the imaging unit 11. This signal wire extends through the inside of the insertion portion 110 to a base end 110B side of the insertion portion 110. If necessary, it is also possible for an optical adapter to be mounted in the imaging unit 11 that is used to adjust the angle of view, the direction of view, and the observation depth and the like of the imaging unit 11.

The illumination unit 12 includes an optical element or the like, and has an illumination light irradiation portion 12A at the distal end 110A of the insertion portion 110, and is a component that illuminates the field of view of the imaging unit 11 using this illumination light.

The bending portion 113 is formed by linking together in the direction of the center axis thereof a line of tubular node rings or bending links (referred to below as “node rings or the like”). The bending portion 113 is flexible and is placed on the distal end 110A side of the insertion portion 110. The bending portion 113 is also able to bend in a direction in which it moves away in the radial direction of the insertion portion 110 from a center axis O of the insertion portion 110. In the endoscope system 1 of the present embodiment, the bending portion 113 is able to cause the distal end 110A side of the insertion portion 110 to bend in two directions in which it moves away in the radial direction from the axis of the insertion portion 110.

As is shown in FIG. 3, a unit main body 24 that is used for mounting the scope unit 100 on the main body unit 20 is provided at the proximal end 110B of the insertion portion 110 of the scope unit 100. Inside the unit main body 24 are provided a light source section 70 which forms a light source for the illumination unit 12, a heat discharge portion 80 which is thermally connected to the light source section 70, a bending drive unit 160 which causes the bending portion 113 to perform a bending operation, and a connector 27 that is used to provide an electrical connection to a connector 26 of the main body unit 20 (described below).

In the present embodiment, an observation device is formed by the aforementioned imaging unit 11, illumination unit 12, and light source section 70. Because the observation device is provided with the imaging unit 11, the illumination unit 12, and the light source section 70, even if internal portions of an examination subject are in darkness, an examiner is still able to illuminate the internal portions of the examination subject and perform a proper examination.

The light source section 70 is provided with an LED 71 that emits illumination light, and a light guide 72 that is positioned such that the illumination light emitted by the LED 71 is irradiated thereon. A structure in which a plurality of optical fibers are bundled together is employed for the light guide 72. The light guide 72 enters inside the insertion portion 110 from the unit main body 24, passes through the interior of the insertion portion 110, and is connected to the illumination unit 12 which is positioned on the distal end 110A side of the insertion portion 110.

The heat discharge unit 80 is formed from a material having superior thermal conductivity such as metal, and has fins 81 and fins 82 that are exposed to the outside of the unit main body 24. Moreover, a portion of the heat discharge unit 80 is inserted inside the unit main body 24 and is thermally connected to the LED 71. The fins 81 and the fins 82 that are exposed to the outside of the unit main body 24 function as a heat sink that allows the heat generated by the LED 71 to escape to the outside of the unit main body 24.

The bending drive unit 160 is provided with a group which is made up of a drive source in the form of a motor 61 and a pulley 62 that is rotated by the motor 61. There is no particular limitation on the method used to link together the motor 61 and pulley 62, and it is sufficient for the pulley 62 to be attached to a shaft of the motor 61. It is also possible for the shaft and the rotation shaft of the pulley 62 to be connected by means of a power transmission component such as a belt or the like.

Two angle wires 14A and 14C that are inserted through the insertion portion 110 and are connected to the node ring or the like on the distal end 110A side of the insertion portion 110 are wound around and connected to the pulley 62. By employing this structure, it is possible to rotate the motor 61 and rotate the pulley 62, and to cause the pair formed by the angle wire 14A and the angle wire 14C to move relatively to the insertion portion 110. As a result, the bending drive unit 160 is able to make the bending portion 113 bend in each of the above described two directions.

As is shown in FIG. 5, a structure storage section 195 in which structure identification information I₁₀₀ is stored that is used to identify the structure of the bending drive unit 160 provided in the scope unit 100 is provided in the scope unit 100.

The structure storage section 195 is formed by a storage element having a semiconductor chip, and, for example, ROM which is non-rewritable non-volatile memory, EPROM which is rewritable non-volatile memory, or flash memory may be employed.

The structure identification information I₁₀₀ that is stored in the structure storage section 195 is provided with bending mode parameters P₁₀₀ that are used to identify the bending mode of the bending drive unit 160 which bends the bending portion 113 of the scope unit 100, and drive parameters P₁₁₀ that are used to cause the scope unit 100 to perform a bending operation. The bending mode parameters P₁₀₀ and the drive parameters P₁₁₀ are stored in the structure storage section 195, for example, in a state of being described in a single setting file F₁₀₀.

The bending mode parameters P₁₀₀ contain information that shows that the scope unit 100 is performing a bending operation using electric power, and information that shows that the scope unit 100 is provided with the bending drive unit 160 that bends the bending portion 113 in two directions. By employing this structure, it can be identified by means of the bending mode parameters P₁₀₀ that the directions in which the bending portion 113 can be bent consist of two directions.

The drive parameters P₁₁₀ contain information that is used to convert an operation input of an examiner which is input into the main body unit 20 into a drive signal that operates the bending drive unit 160. The drive parameters P₁₁₀ in the present embodiment include, specifically, information about a direction P₁₁₁ in which the motor 61 of the bending drive unit 160 is rotated, a rotation amount P₁₁₂ thereof, and a rotation speed P₁₁₃ thereof.

The drive parameters P₁₁₀ are set based on the length and diameter of the insertion portion 110 in order that the bending operation of the bending portion 113 can be appropriately performed.

The direction P₁₁₁ is a parameter that associates together the bend direction of the bending portion 113 and the direction in which the motor 61 is made to rotate. These parameters are determined such that, for example, when a positive voltage is applied to the motor 61, the bending portion 113 bends upwards, and such that, when a negative voltage is applied to the motor 61, the bending portion 113 bends downwards.

Note that the direction P₁₁₁ may also include parameters that, when a command to reverse the bend direction is input while the bending portion 113 is currently performing a bending operation, determine whether or not the bending operation of the bending portion 113 is to be rapidly reversed immediately, or whether the bending portion 113 is to be temporarily stopped first and then reversed slowly.

The rotation amount P₁₁₂ is a parameter that determines the rotation amount of the motor 61, and the rotation speed P₁₁₃ is a parameter that determines the rotation speed of the motor 61.

For each parameter of the drive parameters P₂₁₀ described below, the meaning of the respective parameters, namely, direction, rotation amount, and rotation speed are the same as for the drive parameters P₁₁₀.

Next, the structure of the scope unit 200 will be described. As is shown in FIG. 2, the structure of the scope unit 200 differs from that of the scope unit 100 in that it is provided with an insertion portion 210 which is provided instead of the insertion portion 110 of the scope unit 100, and with the bending drive unit 260 that is provided instead of the bending drive unit 160. In addition, the scope unit 200 is provided with the light source section 70 and the heat discharge unit 80 that have the same structure as those of the scope unit 100.

Hereinafter, the description will center on the points of difference from the scope unit 100.

The insertion portion 210 has a bending portion 213 which is provided instead of the bending portion 113. In the bending portion 213, the structure such as the node rings or the like differs from that of the bending portion 113 in that the node rings or the are orthogonal to the center axis direction of the insertion portion 210 and pivot relatively around two axes which are mutually orthogonal. Because of this, the bending portion 213 is able to bend a distal end 210A of the insertion portion 210 in four directions.

Moreover, because the insertion portion 210 is able to be bent in four directions, as is shown in FIG. 4, an angle wire 14A, an angle wire 14B, an angle wire 14C, and an angle wire 14D that correspond respectively to the four directions in which the bending portion 213 is able to be bent are provided in the bending portion 213.

The bending drive unit 260 is provided with two groups that are each made up of a drive source in the form of the motor 61, and the pulley 62 that is rotated by the motor 61. Of the four angle wires 14A, 14B, 14C, and 14D that are connected to the bending portion 213, the two angle wires 14A and 14C that are positioned facing in the radial direction of the insertion portion 210 are wound around and connected to a first pulley 62A. In addition, the two angle wires 14B and 14D that are positioned facing in directions offset by 90° around the axis of the insertion portion 110 relative respectively to the aforementioned two directions are wound around and connected to a second pulley 62B.

By employing the above described structure, when a first motor 61A and a second motor 61B are each rotated, the corresponding first pulley 62A and second pulley 62B are also rotated. When the first pulley 62A and the second pulley 62B are rotated, the pair made up by the angle wire 14A and the angle wire 14B and the pair made up by the angle wire 14C and the angle wire 14D are moved relatively to the insertion portion 210. As a result of this, the bending drive unit 260 is able to cause the bending portion 213 to bend in directions moving away from the center axis of the insertion portion 210.

Moreover, the connector 27 that is used to provide an electrical connection to the connector 26 of the main body unit 20 (described below) is formed on an external surface of the scope unit 200 in the same way as for the scope unit 100.

Moreover, a structure storage section 295 having the same type of semiconductor chip as the structure storage section 195 is provided in the scope unit 200. Structure identification information I₂₀₀ that is stored in the structure storage section 295 is provided with bending mode parameters P₂₀₀ that are used to identify the bending mode of the bending drive unit 260 that bends the bending portion 213 of the scope unit 200, and drive parameters P₂₁₀ that are used to cause the scope unit 200 to perform a bending operation. The bending mode parameters P₂₀₀ and the drive parameters P₂₁₀ are stored in the structure storage section 295, for example, in a state of being described in a single setting file F₂₀₀.

In the same way as the bending mode parameters P₁₀₀, the bending mode parameters P₂₀₀ contain information that shows that the scope unit 200 is performing a bending operation using electric power, and information that shows that the bending drive unit 260 is provided that bends the bending portion 213 in four directions.

The drive parameters P₂₁₀ contain information that is used to convert an operation input which is made by an examiner into the main body unit 20 into a drive signal that operates the bending drive unit 260. The drive parameters P₂₁₀ in the present embodiment include, specifically, information about a direction P₂₁₁ in which the first motor 61A and the second motor 61B of the bending drive unit 260 are rotated respectively, a rotation amount P₂₁₂ thereof, and a rotation speed P₂₁₃ thereof.

The main body unit 20 is provided such that the scope units 100 and 200 can be removably mounted thereon. When the scope unit 100 or 200 is mounted on the main body unit 20, communication is possible between the scope unit 100 or scope unit 200 and the main body unit 20, and the main body unit 20 controls the scope unit 100 or the scope unit 200.

Moreover, as is shown in FIG. 1, the main body unit 20 is provided with an operating section 21 which is gripped by an examiner and operated, a gripping portion 30 which is provided in the operating section 21 and which is gripped by the examiner, an operation input section 40 which is used to make operation inputs into the bending portion 113, a display unit 50 that displays video signals acquired by the imaging unit 11 as images, and a control unit 90 (see FIG. 5) that controls operations of the scope unit 100 or the scope unit 200 when the scope unit 100 or the scope unit 200 is mounted on the main body unit 20.

Moreover, the operating section 21 has a mounting portion 23 which has a recessed configuration that enables it to hold inside itself the unit main body 24 of the scope unit 100 or the scope unit 200, and has the connector 26 that is used to provide an electrical connection with the scope unit 100 or the scope unit 200 and to perform communication therewith provided on the inner side of the mounting portion 23.

As is shown in FIG. 3, the gripping portion 30 is formed in a bar shape that protrudes from the operating section 21, and has an internal battery 31. The battery 31 is a component that supplies electric drive power to the scope unit 100 or the scope unit 200 and to the display unit 50 provided in the main body unit 20.

The operation input section 40 is a component that inputs the operation direction of the bending portion 113 and also inputs various settings for the display unit 50. The operation input section 40 is provided with a joystick 41 that operates by tilting from a neutral state in four directions, namely, an up direction U, a down direction D, a left direction L, and a right direction R, and with a plurality of push buttons 42.

The joystick 41 has a potentiometer 41A that detects, for example, positions on straight lines that respectively follow two mutually orthogonal axes. By tilting the joystick 41, the resistance value of the potentiometer 41A is changed, and these changes in the resistance value are referred to by the control unit 90 (described below).

Note that, instead of the joystick 41, it is also possible to employ a keypad or buttons or the like that correspond to the directions in which the scope unit 100 or the scope unit 200 is operated.

The display unit 50 is provided with a display screen 51, and with a display control unit 52 that processes video signals sent from the imaging unit 11 so that they are able to be displayed on the display screen 51. It is also possible for what is known as a touch panel type of display to be employed for the display screen 51, and for a structure to be employed in which operation inputs that are made in order to bend the bending portion 113 or the bending portion 213 or operation inputs for the imaging unit 11 are performed via the touch panel of the display screen 51.

As is shown in FIG. 5, the display control unit 52 has a camera control unit 52A (referred to hereinafter as a CCU 52A), and an image recording unit 52B that stores image signals transmitted from the imaging unit 11 to the CCU 52A. The display control unit 52 transmits image information transmitted from the imaging unit 11 provided in the insertion portion 110 of the scope unit 100, for example, as NTSC signals to the display screen 51, and thereby causes images to be displayed on the display screen 51.

As is shown in FIG. 3 and FIG. 4, the control unit 90 is located inside the operating section 21 in the main body unit 20. Moreover, the control unit 90 is electrically connected via the connector 26 to whichever component of the scope unit 100 and the scope unit 200 is mounted on the main body unit 20.

The control unit 90 is also electrically connected inside the main body unit 20 to the display control unit 52 of the display unit 50, and the control unit 90 controls operations of the display unit 50. The control unit 90 is also electrically connected to the operation input section 40, and receives operation inputs that are made when the joystick 41 and buttons 42 are operated.

When the scope unit 100 is mounted on the main body unit 20, the control unit 90 performs communication respectively with the structure storage section 195, the bending drive unit 160, and the light source section 70.

As is shown in FIG. 5, the control unit 90 is provided with a central processing unit 91, a storage unit 92 that is electrically connected to the central processing unit 91, a bend drive circuit 93 that is electrically connected to the central processing unit 91, and a light source drive circuit 94.

The central processing unit 91 operates in accordance with a built-in operating system that is used to drive the endoscope system 1 and controls each one of the storage unit 92, the bend drive circuit 93, and the light source drive circuit 94.

The central processing unit 91 also refers to the resistance value of the potentiometer 41A of the joystick 41 via an I/O circuit (not shown). The central processing unit 91 switches the operation input which is input from the joystick 41 to the control unit 90 between a drive operation input that is used to transmit a drive signal from the bend drive circuit 93 to the bending drive units 160 or 260, and a command input that moves a cursor or the like that is displayed on the display screen 51 and issues commands for coordinates on the display screen 51, and then recognizes each input.

The drive operation input and the command operation input are able to be switched using the button 42, and can be electrically switched based on the information in the structure identification information I₁₀₀ or in the structure identification information I₂₀₀.

Furthermore, the central processing unit 91 controls the display control unit 52 in accordance with a program of an in-built operating system that is used to drive the endoscope system 1 such that a menu screen or images of an examination subject or the like are displayed on the display screen 51.

The storage unit 92 has ROM 92A and RAM 92B. The aforementioned operating system is stored in the ROM 92A. In addition, when the endoscope system 1 is started up, principal components of this operating system are transmitted to the RAM 92B and executed.

In the present embodiment, the RAM 92B is a volatile storage device, and by turning off the power supply of the endoscope system 1, or by removing the scope unit 100 or the scope unit 200 from the main body unit 20, all of the information stored in the RAM 92B is deleted.

Furthermore, the direction in which the joystick 41 is tilted, the angle at which the joystick 41 is tilted, and the speed at which the joystick 41 is tilted are temporarily stored in the RAM 92B.

The structure identification information I₁₀₀ and I₂₀₀ that are stored in the structure storage section 195 of the scope unit 100 and the structure storage section 295 of the scope unit 200 are read to the RAM 92B when the endoscope system 1 is started up.

In the present embodiment, the structure identification information I₁₀₀ or the structure identification information I₂₀₀ is stored in the RAM 92B as a result of the setting files F₁₀₀ or F₂₀₀ that are stored in the structure storage section 195 or the structure storage section 295 being transmitted to the RAM 92B.

Note that it is possible for rewritable nonvolatile memory to be employed for the RAM 92B. In this case, even if the power supply of the endoscope system 1 is shut down, the structure identification information can still be retained. In addition, it is also possible to employ a structure in which, when the scope unit 100 and the scope unit 200 are switched and mounted on the main body unit 20 and used, if necessary, the structure identification information can be read from the structure storage section 195 or the structure storage section 295 and written to the RAM 92B.

The bend drive circuit 93 is a circuit that generates a bend drive signal in order to drive the bending drive unit 160 or the bending drive unit 260. The bend drive circuit 93 converts the information about the tilt direction of the joystick 41, the tilt angle of the joystick 41, and the tilt speed of the joystick 41 based on the structure identification information I₁₀₀ and the structure identification information I₂₀₀ which are each stored in the RAM 92B, and creates a bend drive signal that is used to drive the bending drive unit 160 or the bending drive unit 260, and transmits this bend drive signal to the bending drive unit 160 or the bending drive unit 260.

Note that when these setting files do not exist in the RAM 92B, the bend drive circuit 93 does not operate. At this time, the power supply of the portion of the control unit 90 that is associated with the bend drive circuit 93 can be turned off.

The light source drive circuit 94 is a circuit that is used to drive the light source section 70. In the light source drive circuit 94, voltage for the drive power that drives the LED 71, and light source drive signal that commands about the current and drive waveform are created respectively based on the structure identification information I₁₀₀ and I₂₀₀ stored respectively in the RAM 92B, and these drive signals are supplied to the LED 71.

Note that the light source drive circuit 94 has a prescribed light source drive signal that is used to drive the light source section 70 when none of the above described structure identification information is present in the RAM 92B. In addition, the light source drive circuit 94 causes the LED 71 to emit light based on this prescribed light source drive signal when the structure identification information is not present.

Operations when the endoscope system 1 of the present embodiment having the above described structure is in use will now be described.

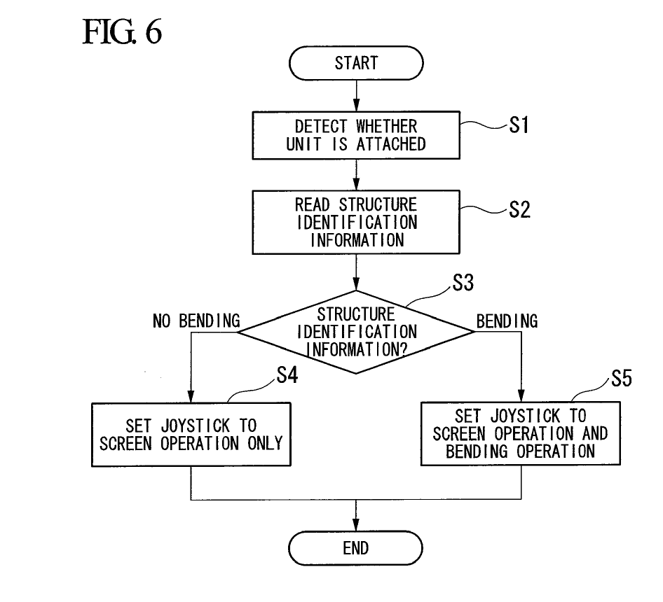

FIG. 6 through FIG. 8 are flowcharts for illustrating operations of the endoscope system 1 of the present embodiment.

Firstly, a mounting step in which the scope unit 100 or the scope unit 200 is mounted on the main body unit 20 will be described with reference made to FIG. 6.

Step S1 is a step in which unit mounting detection is performed which detects that a scope unit has been mounted on the main body unit 20.

In step S1, an examiner mounts, for example, the scope unit 100 on the mounting portion 23 of the main body unit 20. As a result of this, the connector 27 provided in the scope unit 100 is connected to the connector 26 which is provided in the main body unit 20. At this time, both the bending drive unit 160 and the light source section 70 are electrically connected to the control unit 90. Here, the bending drive unit 160 is connected to the bend drive circuit 93 of the control unit 90, while the LED 71 is connected to the light source drive circuit 94 of the control unit 90.

The light source drive circuit 94 transmits a signal showing that a scope unit such as the scope unit 100 or the scope unit 200 has been connected to the central processing unit 91 when the LED 71 is connected. As a result of this, it is detected in the central processing unit 91 that a scope unit has been mounted on the main body unit 20.

At this point, step S1 ends and the routine moves to step S2.

Step S2 is a step in which structure identification information about the scope unit connected to the main body unit 20 is read to the main body unit 20.

In step S2, when the scope unit 100 is mounted on the main body unit 20, the setting file F100 in which the structure identification information I₁₀₀ is described is read to the RAM 92B of the control unit 90 from the structure storage section 195 of the scope unit 100.

At this point, step S2 ends and the routine moves to step S3.

Step S3 is a step in which a determination is made as to whether or not structure identification information exists.

In step S3, firstly, whether or not the operation to read the structure identification information to the RAM 92B was successful and whether or not the structure identification information was corrupt are confirmed.

Note that in the present embodiment, the structure identification information I₁₀₀ and I₂₀₀ are stored respectively in the scope unit 100 and the scope unit 200. However, there are cases when structure identification information is not provided in a scope unit that does not require a bending operation. In such cases, it is also possible to detect that structure identification information is not provided by detecting that there has been no response from a scope unit even when a predetermined timeout time which is stored in the ROM 92A has elapsed.

In cases when structure identification information that shows that the bend drive portion 160 or the bend drive portion 260 is present does not exist, step S3 is ended and the routine moves to step S4.

In the present embodiment, the fact that the bending mode parameter P₂₀₀ is an electrical bending type is stored in the structure identification information I₁₀₀ and I₂₀₀ of the scope units 100 and 200. Because of this, in step S3, it is determined that the scope unit is a bend-driven scope unit and the routine moves from step S3 to step S5.

Step S4 is a step in which the operation performed by the control unit 90 in response to an input operation input on the joystick 41 is set.

In step S4, the control unit 90 is set such that it is confirmed that the operation input that is input on the joystick 41 is a command operation input intended to select one of the alternatives displayed on the display screen 51, or to specify a portion of a graphic displayed on the display screen 51. At this time, the operation by an examiner to tilt the joystick 41 is referred to by the control unit 90. In addition, in conjunction with the operation of the joystick 41 the control unit 90 moves a cursor or the like that is displayed on the display screen 51 in order to indicate a point on the display screen 51.

As a result, step S4 is ended and the operation of the joystick 41 is used for the operation of the display screen 51.

Step S5 is a step in which the operation performed by the control unit 90 in response to an input operation input on the joystick 41 is set.

In step S5, the control unit 90 switches between drive operation inputs that are makes the bend drive portion 160 provided in the scope unit 100 operate, and the above described command operation inputs, and recognizes operation inputs that are input on the joystick 41. At this time, the operations of the joystick 41, for example, can be switched alternately by pressing the button 42.

As a result of this, step S5 is ended and the operation of the joystick 41 is used for both the operation of the bending drive unit 160 and the operation of the display screen 51.

At this point, the mounting step in which the scope unit 100 or the scope unit 200 is mounted on the main body unit 20 is ended.

After the mounting step has ended, a mode selection step in which the bending mode that is used to bend the scope unit is selected is then started in the endoscope system 1.

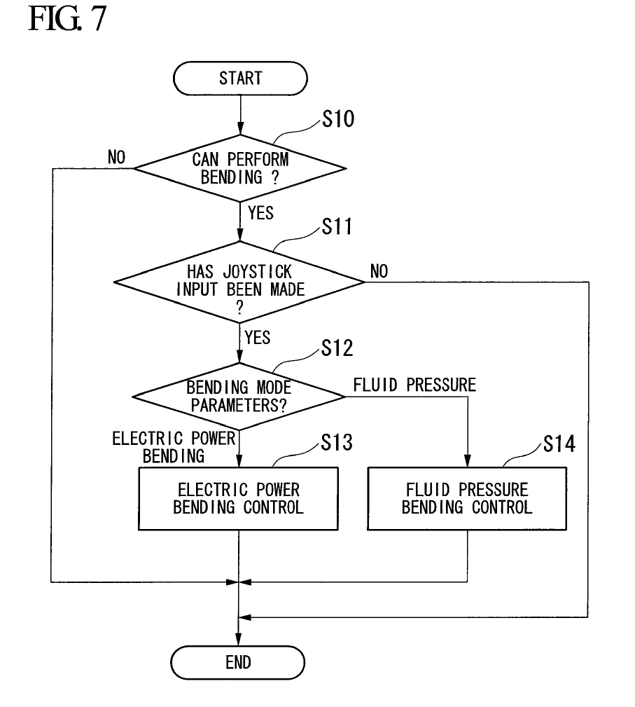

As is shown in FIG. 7, step S10 is a step in which it is determined whether or not the scope unit which is mounted on the main body unit 20 shown in FIG. 2 is able to perform a bending operation.

In step S10, if the bending mode parameters P₁₀₀ or P₂₀₀ are present based on the structure identification information I₁₀₀ or the structure identification information I₂₀₀ stored in the RAM 92B in step S2, then step S10 is ended and the routine moves to step S11.

Note that, for example, when a scope unit that does not perform a bending operation is mounted on the main body unit 20, because bending mode parameter information is not included in the structure identification information, step S10 is ended and the parameter selection step is also ended.

Step S11 is a step in which it is determined whether or not an input operation has been performed by an examiner on the joystick 41.

In step S11, the control unit 90 refers to the resistance value of the potentiometer 41A of the joystick 41, and determines from the resistance value of the potentiometer 41A whether or not the joystick 41 is in a neutral position. If an operation input is made by an examiner on the joystick 41, because the joystick 41 is thereby tilted so as to become positioned in a different position from the neutral position, the resistance value of the potentiometer 41A is different from when the joystick 41 is in a neutral position.

In the control unit 90, if the resistance value when the joystick 41 is in a neutral position is different from the resistance value of the potentiometer 41A when step S11 is being executed, then step S11 is ended and the routine moves to step S12.

Note that if there has been no operation input to the joystick 41 by an examiner, the joystick 41 is in a neutral position and, once it has been determined by the control unit 90 that the joystick 41 is in a neutral position, step S11 is ended and the mode selection step is also ended.

Step S12 is a step in which the bending mode of the scope unit is selected.

In step S12, based on the structure identification information read to the RAM 92B, the bending mode of the scope unit is selected in the control unit 90. In the present embodiment, the fact that the bending mode of the scope unit 100 is an electrical power bending mode is stored in the bending mode parameter P₁₀₀ of the structure identification information I₁₀₀ of the scope unit 100. Because of this, in step S12 it is determined that the bending mode of the scope unit 100 is an electric power bending mode, and step S12 is then ended and the routine moves to step S13.

Moreover, in step S12, if a bending mode other than electric power bending is stored in the bending mode parameters, step S12 is ended and the routine moves to step S14. In the present embodiment, an example of a bending mode other than an electric power bending mode that may be considered is the providing in a scope unit of a bending drive unit that causes the bending portion to perform a bending operation by means of fluid pressure. The structure and operation of a scope unit provided with a bending mode that is based on fluid pressure is described below.

Step S13 is a step in which the scope unit is operated in accordance with an electric power bending mode.

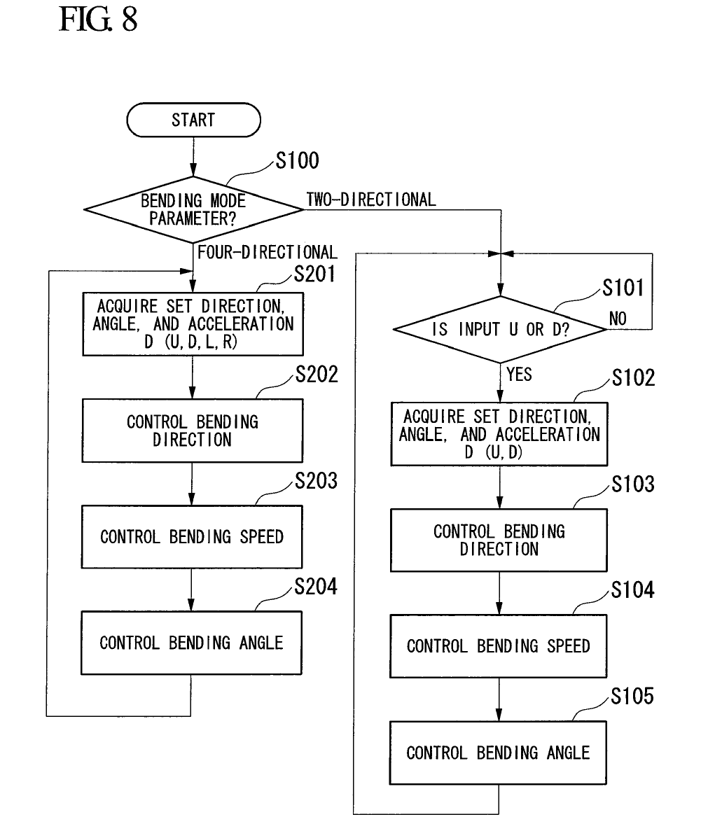

FIG. 8 is a flowchart showing the operation of the endoscope system 1 in step S13 in greater detail.

In step S13 shown in FIG. 8, firstly step S100 is started. In step S100, the control unit 90 refers to those parameters from among the structure identification information I₁₀₀ that has been read to the RAM 92B that cause the scope unit 100 or the scope unit 200 to perform a bending operation.

If the scope unit 100 is mounted on the main body unit 20, then the fact that the bending movements of the bending portion 113 performed by the bending drive unit 160 of the scope unit 100 move in two directions is stored in the bend mode parameters P₁₀₀ in the structure identification information I₁₀₀. As a result, step S100 is ended and the routine moves to step S101.

Note that if the scope unit 200 is mounted on the main body unit 20, then the fact that the bending operations of the bending portion 213 which are driven by the bending drive unit 260 of the scope unit 200 move in four directions is stored in the bend mode parameters P₂₀₀ in the structure identification information I₂₀₀. As a result, step S100 is ended and the routine moves to step S201.

The bending operation step which causes the bending portion 113 or the bending portion 213 to perform a bending operation will now be described with reference made to FIG. 8.

Step S101 is a step in which the type of operation input made by an examiner using the joystick 41 is detected.

In step S101, the control unit 90 refers to the resistance value of the potentiometer 41A that is stored in the RAM 92B. If the joystick 41 is tilted in an upward direction U or in a downward direction D, step S101 is ended and the routine moves to step S102.

If the joystick 41 is tilted in a direction L or in a right direction R, then step S101 is ended and step S101 is started once again. Note that if the left direction L or the right direction R is input by an examiner using the joystick 41 simultaneously with an up direction U or down direction D, then this is dealt with as if there has only been an input in the up direction U or the down direction D, and the routine moves to step S102.

Step S102 is a step in which, based on the resistance value of the potentiometer 41A stored in the control unit 90, the direction, angle, and acceleration of the tilting of the joystick 41 are acquired.

In step S102, the direction, angle, and acceleration of the tilting of the joystick 41 are written in the RAM 92B.

At this point, step S102 is ended and the routine moves to step S103.

Step S103 is a step in which a drive signal is generated that causes the bending drive unit 160 to operate such that the bending direction of the bending portion 113 of the scope unit 100 is controlled based on the information relating to the direction in which the joystick 41 is tilted from among the information written in the RAM 92B in step S102.

In step S103, of the information stored in the RAM 92B, the bend drive circuit 93 of the control unit 90 refers to the direction in which the joystick 41 is tilted, and to the drive parameters P₁₁₀ of the structure identification information I₁₀₀ which is provided in the bending drive unit 160 of the scope unit 100. The bend drive circuit 93 converts the direction in which the joystick 41 is tilted to information showing the direction in which the motor 61 is to be rotated based on the drive parameters P₂₁₀, and generates a drive signal causing the motor 61 to be rotated.

At this point, step S103 is ended and the routine moves to step S104.

Step S104 is a step in which a drive signal is generated that causes the bending drive unit 160 of the scope unit 100 to operate such that the bending speed of the bending portion 113 is controlled based on the information relating to the acceleration at which the joystick 41 is tilted from among the information written in the RAM 92B in step S102.

In step S104, of the information stored in the RAM 92B, the bend drive circuit 93 of the control unit 90 refers to the acceleration at which the joystick 41 is tilted, and to the drive parameters P₁₁₀ of the structure identification information I₁₀₀ which is provided in the bending drive unit 160 of the scope unit 100. The bend drive circuit 93 converts the direction in which the joystick 41 is tilted to information showing the speed at which the motor 61 is to be rotated based on the drive parameters P₁₁₀, and generates a drive signal causing the motor 61 to be rotated.

At this point, step S104 is ended and the routine moves to step S105.

Step S105 is a step in which a drive signal is generated that causes the bending drive unit 160 of the scope unit 100 to operate such that the bending angle of the bending portion 113 is controlled based on the information relating to the angle at which the joystick 41 is tilted from among the information written in the RAM 92B in step S102.

In step S105, of the information stored in the RAM 92B, the bend drive circuit 93 of the control unit 90 refers to the angle at which the joystick 41 is tilted, and to the drive parameters P₁₁₀ of the structure identification information I₁₀₀ which is provided in the bending drive unit 160 of the scope unit 100. The bend drive circuit 93 converts the angle at which the joystick 41 is tilted to a rotation amount to which the motor 61 is to be rotated based on the drive parameters P₁₁₀, and generates a drive signal causing the motor 61 to be rotated.

As a result of the processing of processing of steps S103, S104, and S105, drive signals are generated respectively that specify the direction in which the motor 61 is rotated, the rotation speed, and also the rotation amount thereof. The bend drive circuit 93 causes the motor 61 of the bending drive unit 160 to be driven based on the drive signals generated in steps S103, S104, and S105. At this point, step S105 is ended and the routine returns to step S101.

In this manner, by repeating a loop from step S101 to step S105, the motor 61 of the bending drive unit 160 is made to rotate so as to correspond to the position to which the joystick 41 has been tilted by the examiner, and the bending portion 113 is thereby made to perform a bending operation. The loop from step S101 to step S105 is ended by an end-interrupt generated in end processing which is performed in order to shut off the power supply of the endoscope system 1, or by a switch-interrupt which shuts off the supply of power to the scope unit 100 in order for the scope unit 100 to be switched with another scope unit.

Hereinafter, an operation of the endoscope system 1 when it is determined in the above described step S100 that the bending mode of the scope unit is four-directional bending, namely, when the scope unit 200 is mounted in the main body unit 20 will be described with reference made to FIG. 8.

Step S201 is a step in which, based on the resistance value of the potentiometer 41A stored in the control unit 90, the direction in which the joystick 41 is tilted, as well as the angle and the acceleration thereof are acquired.

In step S201, the direction in which the joystick 41 is tilted, as well as the angle and the acceleration thereof are written in the RAM 92B.

At this point, step S201 is ended and the routine moves to step S202.

Step S202 is a step in which a drive signal is generated that causes the bending drive unit 260 to operate such that the bending direction of the bending portion 213 of the scope unit 200 is controlled based on the information relating to the direction in which the joystick 41 is tilted from among the information written in the RAM 92B in step S202.

In step S202, of the information stored in the RAM 92B, the bend drive circuit 93 of the control unit 90 refers to the direction in which the joystick 41 is tilted, and to the drive parameters P₂₁₀ of the structure identification information I₂₀₀ which is provided in the bending drive unit 260 of the scope unit 200. The bend drive circuit 93 converts the direction in which the joystick 41 is tilted to information showing which of the first motor 61A and the second motor 61B is the motor to be driven, and information showing the directions in which the motor 61A and the motor 61B are to be rotated respectively based on the drive parameters P₂₁₀, and generates a drive signal causing the motor 61 to be rotated.

At this point, step S202 is ended and the routine moves to step S203.

Step S203 is a step in which a drive signal is generated that causes the bending drive unit 260 to operate such that the bending speed of the bending portion 213 of the scope unit 200 is controlled based on the information relating to the acceleration at which the joystick 41 is tilted from among the information written in the RAM 92B in step S203.

In step S203, of the information stored in the RAM 92B, the bend drive circuit 93 of the control unit 90 refers to the acceleration at which the joystick 41 is tilted, and to the drive parameters P₂₁₀ of the structure identification information I₂₀₀ which is provided in the bending drive unit 260 of the scope unit 200. The bend drive circuit 93 converts the direction in which the joystick 41 is tilted into the speed at which the motor 61 is to be rotated based on the drive parameters P₂₁₀, and generates a drive signal causing the motor 61 to be rotated.

At this point, step S203 is ended and the routine moves to step S204.

Step S204 is a step in which a drive signal is generated that causes the bending drive unit 260 to operate such that the bending angle of the bending portion 213 of the scope unit 200 is controlled based on the information relating to the angle at which the joystick 41 is tilted from among the information written in the RAM 92B in step S203.

In step S203, of the information stored in the RAM 92B, the bend drive circuit 93 of the control unit 90 refers to the angle at which the joystick 41 is tilted, and to the drive parameters P₂₁₀ of the structure identification information I₂₀₀ which is provided in the bending drive unit 260 of the scope unit 200. The bend drive circuit 93 converts the angle at which the joystick 41 is tilted to a rotation amount to which the motor 61 is to be rotated based on the drive parameters P₂₁₀, and generates a drive signal causing the motor 61 to be rotated.

As a result of the processing of processing of steps S202, S203, and S204, drive signals are generated respectively that specify the direction in which the first motor 61A and the second motor 61B are rotated, the rotation speed, and also the rotation amount thereof. The bend drive circuit 93 causes the first motor 61A and the second motor 61B of the bending drive unit 260 to be driven based on the drive signals generated in steps S202, S203, and S204. At this point, step S204 is ended and the routine returns to step S201.

In this manner, by repeating a loop from step S201 to step S204, the first motor 61A and the second motor 61B of the bending drive unit 260 are made to rotate in synchronization with the position to which the joystick 41 has been tilted by the examiner, and the bending portion 213 is thereby made to perform a bending operation. The loop from step S201 to step S204 is ended by an end-interrupt generated in end processing which is performed in order to shut off the power supply of the endoscope system 1, or by a switch-interrupt which shuts off the supply of power to the scope unit 200 in order for the scope unit 200 to be switched with another scope unit.

As has been described above, according to the endoscope device of the present embodiment, based on the structure identification information stored in the structure storage section 195 or the structure storage section 295 which are provided in the scope unit 100 or the scope unit 200, it is possible to generate drive signals in the bend drive circuit 93 for each one of the scope units that are switched and used, and transmit these from the main body unit 20 to the scope unit 100 or the scope unit 200. Accordingly, even if a plurality of scope units that each have a different structure are switched and used in the main body unit, an operation input into the main body unit can still be accurately reflected to the scope unit.

Moreover, in step S2, the control unit 90 reads the structure identification information from the scope unit 100 or the scope unit 200, and is stored in the RAM 92B. Moreover, in step S3, based on the structure identification information I₁₀₀ and I₂₀₀, the control unit 90 receives operations for which the operation input was input into the joystick 41 of the operation input section 40, and differentiates between drive operation inputs that are used to drive the bending portion 113 or the bending portion 213, and command operation inputs that indicate coordinates on the display unit 50, and also recognizes these respective inputs. Because of this, it is possible to set an appropriate method of use for the joystick 41 in accordance with the structure of the scope unit.

Moreover, the bending drive unit 160 is provided in the scope unit 100, and the bending drive unit 260 is provided in the scope unit 200. Because a drive unit that is used to make a bending portion perform a bending operation is provided on the scope unit side in this manner, heavy objects are not placed unnecessarily on the main body unit 20 side, and the weight of the main body unit 20 can be lightened.

Moreover, the structure identification information that is stored in the structure storage section 195 of the scope unit 100 and in the structure storage section 295 of the scope unit 200 is different for the structure of each scope unit. Because of this, even if the scope unit is switched while the endoscope system 1 is being used, there is no mixing up of the structure identification information, and the control unit 90 is able to create drive vibration that corresponds to the scope unit.

Moreover, the scope unit is able to identify directions in which bending is possible as well as the bending mode based on the bending mode parameters P₁₀₀ and P₂₀₀. As a result of this, because it is no longer necessary for an examiner to manually identify directions in which bending is possible or the bending mode, operating the endoscope system is made easy.

Moreover, the structure identification information also has parameters that are used to bend the bending portion. Because of this, the control unit 90 is able to convert the operation in the bend drive circuit 93 in which the joystick 41 is tilted into a drive signal for the motors of the bending drive unit 160 or the bending drive unit 260. Accordingly, an operation input in the main body unit is accurately reflected to the scope unit.

### Variant Example 1

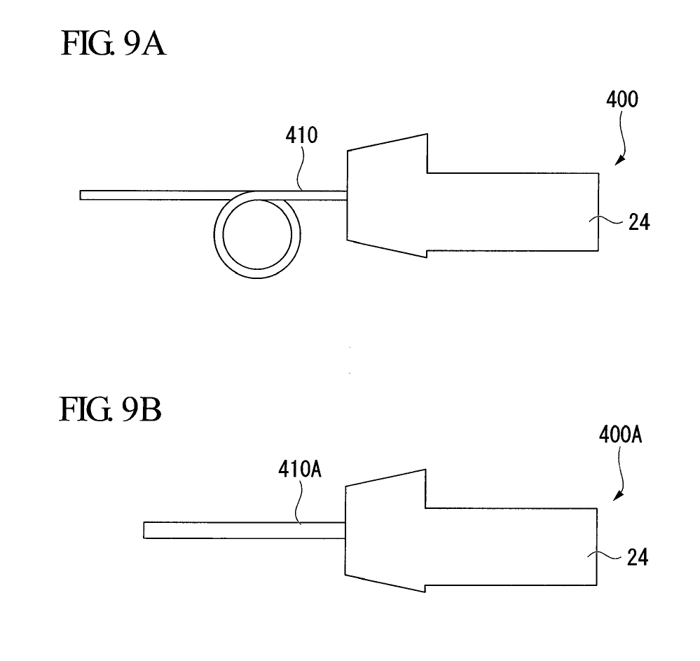

Hereinafter, the structure of variant example 1 of the endoscope system 1 of the present embodiment will be described with reference made to FIG. 9A through FIG. 9B.

FIG. 9A is a side view showing a scope unit that is able to be mounted on the endoscope system 1 of the present embodiment. FIG. 9B is a side view showing another scope unit that is able to be mounted on the endoscope system 1 of the present embodiment.

A scope unit 400 shown in FIG. 9A is provided with a unit main body 24 that is mounted on the mounting portion 23 of the main body unit 20 shown in FIG. 2, and with a second insertion portion 410 that one end is fixed to the unit main body 24. The second insertion portion 410 is flexible, however, unlike the scope unit 100 and the scope unit 200, there is no mechanism for bending the second insertion portion 410.

Moreover, although omitted from the drawings, the scope unit 400 is also provided with the imaging unit 11 of the scope unit 100, a second observation device that are formed in the same way as the illumination unit 12 and the light source section 70, and a second light source section having a second illumination unit and a second light source section.

The endoscope system 1 of the present embodiment can also be provided, in addition to the scope unit 100 or the scope unit 200, with a scope unit such as the scope unit 400 as a second scope unit.

The structure of the scope unit 400A shown in FIG. 9B differs from that of the above described scope unit 400 in that it is provided with an insertion portion 410A instead of the second insertion portion 410. The insertion portion 410A is formed in a rigid cylinder shape. The endoscope system 1 of the present embodiment can be provided with the scope unit 400A.

When the scope unit 400 is fitted in the main body unit 20 instead of the scope unit 100 or the scope unit 200, the control unit 90 receives operations that are input via the joystick 41 of the operation input section 40. Based on the structure identification information of the scope unit 400, the control unit 90 recognizes such operation inputs as a command operation input indicating coordinates on the display unit 50.

### Variant Example 2

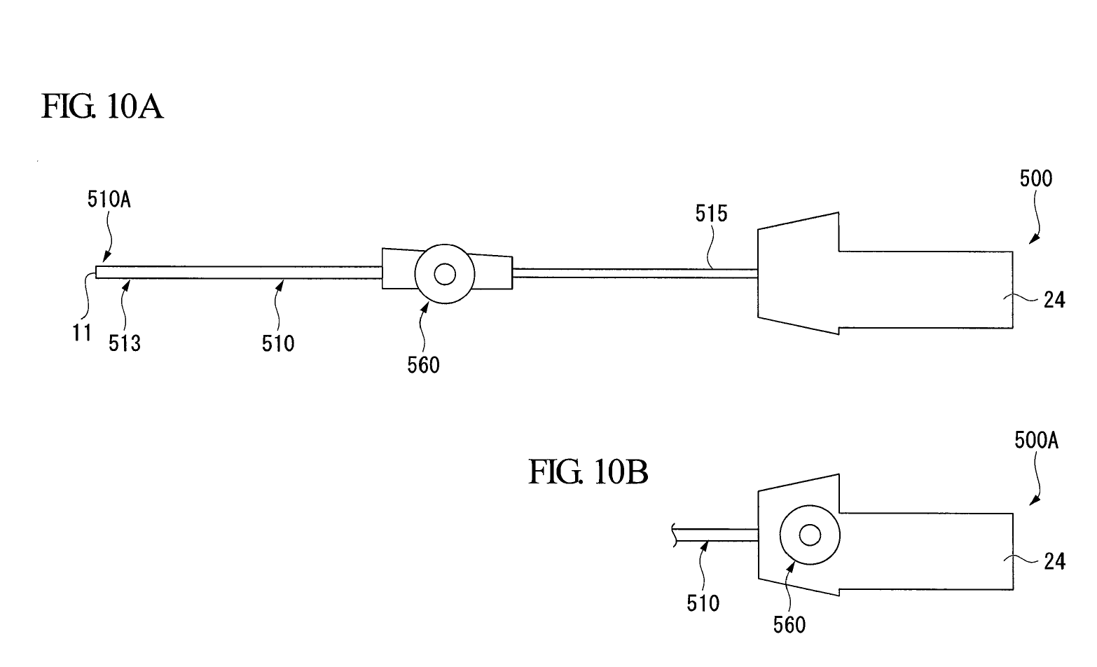

Hereinafter, the structure of variant example 2 of the endoscope system 1 of the present embodiment will be described with reference made to FIG. 10A through FIG. 10B.

FIG. 10A is a side view showing a scope unit that is able to be mounted on the endoscope system 1 of the present embodiment. FIG. 10B is a side view showing another scope unit that is able to be mounted on the endoscope system 1 of the present embodiment.

A scope unit 500 shown in FIG. 10A has a structure that differs from that of the above described scope unit 400 in that it is provided with a second insertion portion 510 that is provided instead of the above described second insertion portion 410, and with a connecting cord 515 and an angle knob 560 that are provided between the second insertion portion 510 and the unit main body 24.

In this variant example, in the same way as in the above described insertion portion 210, an angle wire 14A, an angle wire 14B, an angle wire 14C, and an angle wire 14D are provided within the second insertion portion 510. The angle wire 14A, the angle wire 14B, the angle wire 14C, and the angle wire 14D are pulled by the angle knob 560. As a result, a bending portion 513 on a distal end 510A side of the second insertion portion 510 performs a bending operation.

Although omitted from the drawings, the light guide 72 and a signal wire that is connected to the imaging unit 11 are placed on the connecting cord 515, and a second observation device is constructed in the same way as the above described scope unit 400.

In the present variant example, instead of performing an operation to cause the above described motor 61 to pivot using electrical power, an examiner is able to pull the angle wire 14A, the angle wire 14B, the angle wire 14C, and the angle wire 14D respectively so as to manually cause the angle knob 560 to pivot and thereby cause the bending portion 213 at the distal end of the second insertion portion 510 to perform a bending operation. It is also possible for this type of scope unit to be provided in the endoscope system 1 as a second scope unit together with the scope unit 100 or the scope unit 200.

Moreover, as is shown in FIG. 10B, in the scope unit 500A, the angle knob 560 is mounted on the unit main body 24. In this case, because the positions of the main body unit 20 and the angle knob 560 are close together, an examiner can operate the angle knob 560 while supporting the main body unit 20 with both hands.

### Variant Example 3

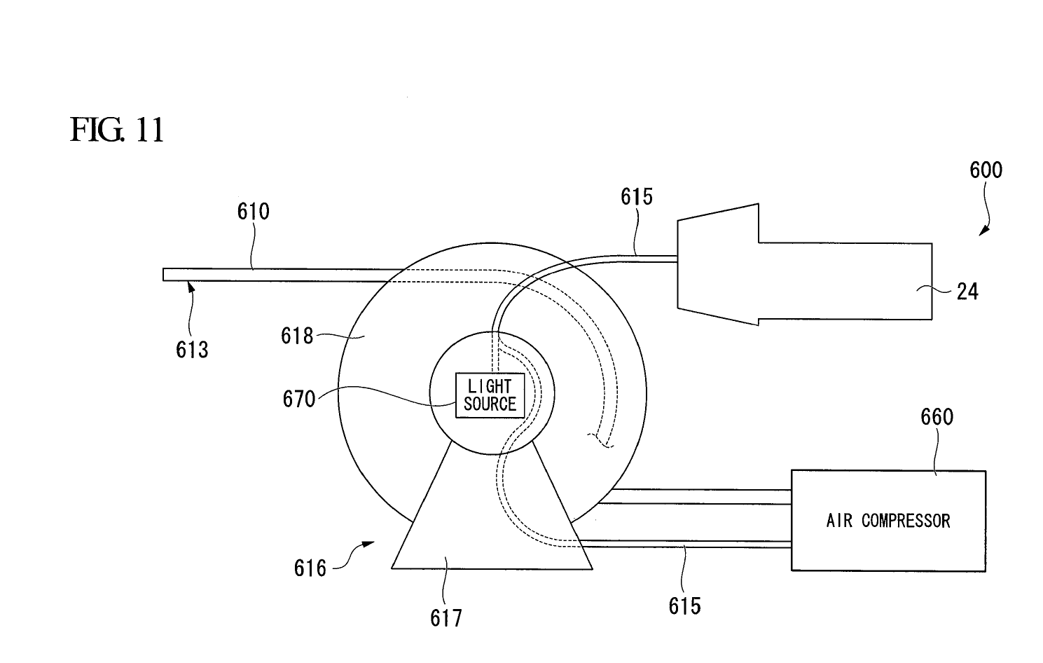

Hereinafter, the structure of variant example 3 of the endoscope system 1 of the present embodiment will be described with reference made to FIG. 11. FIG. 11 is a side view showing a scope unit that is able to be mounted on the endoscope system 1 of the present embodiment.

A scope unit 600 shown in FIG. 11 is a scope unit which, unlike the above described scope unit 100 and scope unit 200, performs a bending operation using fluid pressure. The scope unit 600 is provided with the unit main body 24 which is able to be mounted on the main body unit 20, a connecting cord 615 that has one end fixed to the unit main body 24, and an insertion unit 616 that is connected to the connecting cord 615.

The insertion unit 616 is provided with a second insertion portion 610 that is inserted into an examination subject, a rotating reel 618 around which the second insertion portion 610 is wound, a supporting portion 617 that supports the rotating reel 618, and an air compressor 660 that is connected to the second insertion portion 610 and the connecting cord 615.

The second insertion portion 610 of the present embodiment has a second bending portion 613 in which an actuator is made to perform an extending or contracting movement by means of air supplied from the air compressor 660. In addition, the second insertion portion 610 of the present embodiment is able to change the size of the bend in the second bending portion 613 in accordance with the quantity of air supplied to the actuator.

Moreover, in the present variant example, the scope unit 600 is provided with a second light source section 670 which is provided in the insertion unit 616 instead of the light source section 70 provided inside the unit main body 24. Operations of the second light source section 670 are controlled by the light source drive circuit 94 of the control unit 90 in the same way as the light source section 70.

The scope unit 600 of the present variant example is provided with a structure storage section 695 inside the unit main body 24. Structure identification information that is used to identify the structure of the scope unit 600 is stored in the structure storage section 695. This identification information includes bending mode parameters that show that the scope unit 600 has a bending mode other than electric power bending, and drive parameters that are used to operate the air compressor 660 provided in the scope unit 600.

Here, the drive parameters that are used to operate the air compressor 660 are information that includes parameters that are used to convert the resistance value of the potentiometer 41A of the operation input section 40 into drive signals in the bend drive circuit 93, and a communication procedure that is used to transmit these drive signals from the bend drive circuit 93 to the air compressor 660.

When the scope unit 600 of the present variant example is mounted on the main body unit 20, in step S12 shown in FIG. 7, a bending mode other than electric power bending is selected and the routine moves to step S14.

Step S14 is a step in which the air compressor 660 of the scope unit 600 is driven by means of fluid pressure bending control.

In step S14, in the same way as the above described step S13 the resistance value of the potentiometer 41A is temporarily stored in the RAM 92B of the control unit 90. Furthermore, the bend drive circuit 93 refers to the information stored in the RAM 92B, and converts the resistance value of the potentiometer 41A into a drive signal for driving the air compressor 660 based on the structure identification information, and transmits this to the compressor 660. As a result, the second bending portion 613 can be made to perform a bending operation in the direction in which the joystick 41 has been tilted.

In the same way as the above described scope unit 400, the scope unit 400A, the scope unit 500, and the scope unit 500A, the scope unit 600 of the present variant example can also be provided in the endoscope system 1 as a second scope unit in addition to the scope unit 100 or the scope unit 200.

In the present variant example, the structure of the air compressor 660 that causes the second insertion portion 610 to bend is considerably different from the structure of the above described bending drive unit 160 of the scope unit 100. However, it is possible to generate a suitable drive signal to drive the air compressor 660 based on the structure identification information, and thereby drive the air compressor 660. In this manner, even if the scope unit 600 of the present variant example (i.e., a second scope unit) is mounted as a replacement and used in the endoscope system 1 of the present embodiment, these scope units can still be operated favorably.

### Variant Example 4

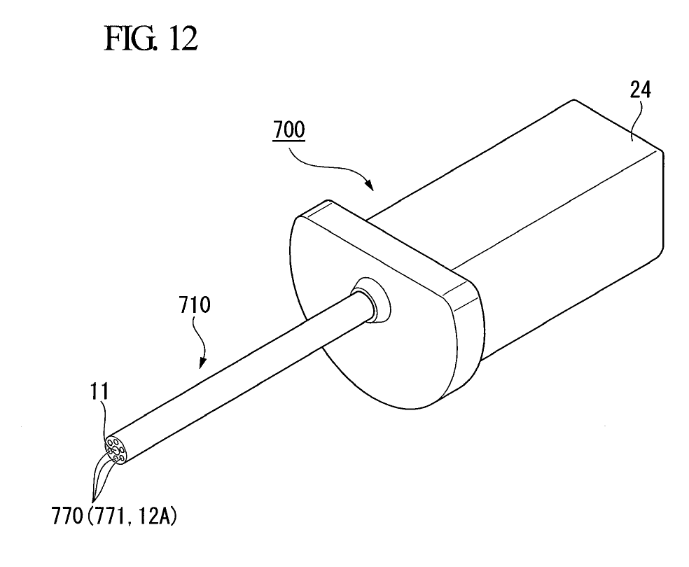

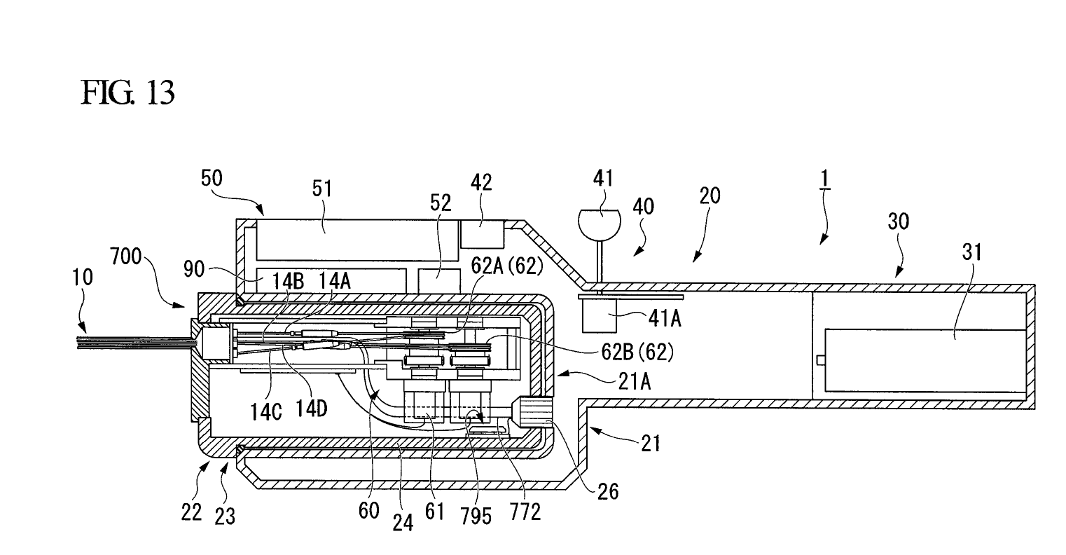

Hereinafter, the structure of variant example 4 of the endoscope system 1 of the present embodiment will be described with reference made to FIG. 12 and FIG. 13. FIG. 12 is a perspective view showing a scope unit that is able to be mounted on the endoscope system 1 of the present embodiment. FIG. 13 is a cross-sectional view showing the structure of a portion of a scope unit that is able to be mounted on the endoscope system 1 of the present embodiment.

As is shown in FIG. 12 and FIG. 13, the structure of the scope unit 700 of the present variant example differs from that of the scope unit 200 in that it is provided with a second insertion portion 710 that is provided instead of the insertion portion 210, and with an LED 771 (i.e., a second light source) that is provided instead of the LED 71. Note that the scope unit 700 has the same type of bending drive unit as the above described bending drive unit 260.

The LED 771 has a plurality of LED that are provided in the vicinity of the irradiation portion 12A of the second insertion portion 710. A power supply wire 772 that is used to transmit electric drive power to the LED 771 is laid inside the second insertion portion 710. The power supply wire 772 is electrically connected to the light source drive circuit 94 of the main body unit 20 via the connector 27 and the connector 26.

A structure storage section 795 that is used to store structure identification information is provided in the unit main body 24 of the scope unit 700. In addition to drive parameters that are used to drive the bending drive unit 260, illumination parameters P₇₂₀ that are used to drive the light source section 770 are also stored in the structure storage section 795 as structure identification information.

In order for the light emitting method which is used to cause the LED 771 to emit light to be selected on the display screen 51, the illumination parameters P₇₂₀ which are used to drive the light source section 770 include light emitting method list information P₇₂₁ that is displayed on the menu screen, and illumination drive parameters P₇₂₂ that are used to generate drive signals in the light source drive circuit 94 in correspondence with this list information. The illumination drive parameters P₇₂₂ which are used to generate the drive signals that drive the light source section 770 may be the voltage and current and the like that are supplied to the LED 771, or may be time width information that shows the light emission timings when the LED 771 is made to emit light intermittently.

When the scope unit 700 is mounted on the main body unit 20, the structure identification information is read from the structure storage section 795 to the RAM 92B. As a result of this, in the same way as in the above described scope unit 200, a drive signal that causes the bending portion 213 to perform a bending operation can be generated by the bending drive circuit 93. Furthermore, a list of light emission methods for causing the LED 771 to emit light are displayed on the display screen 51 based on the list information P₇₂₁, and the light emission method for the LED 771 can be selected on the display screen by tilting the joystick 41.

In this manner, according to the scope unit 700 of the present variant example, the illumination parameters P₇₂₀ that include the list information P₇₂₁ and the illumination drive parameters P₇₂₂ are provided in the structure identification information. Because of this, when scope units having different structures are mounted on the main body unit 20 and used, it is possible to select the light emission method with which the scope unit is equipped by viewing the display screen 51. As a result, even if a plurality of scope units that are equipped with mutually different light emission methods for each respective scope unit are switched and used, it is easy to understand the usage method for each scope unit.

Note that, in the same way as the above described scope unit 400, the scope unit 400A, the scope unit 500, the scope unit 500A, and the scope unit 600, the scope unit 700 of the present variant example can also be provided in the endoscope system 1 as a second scope unit in addition to the scope unit 100 or the scope unit 200. In this case, even if the second insertion portion 710 has an extended length, there is less transmission loss in the illumination light when the illumination light from the LED 71 is transmitted using the light guide 72 compared with a structure in which the illumination light is transmitted to the irradiation portion 12A, and it is possible to form a more narrow second insertion portion 710.

Moreover, as examples of scope units having differently structured illumination units, in addition to the scope unit 700, there are scope units, for example, that employ light sources other than LED. For example, scope units that employ a halogen lamp or laser diode (LD) as a light source can be considered. In this case as well, by storing structure identification information that includes a light emission method and parameters that correspond to a halogen lamp or laser diode (LD) in the structure storage section, the control unit 90 of the main body unit 20 is able to control the respective light sources.

### Variant Example 5

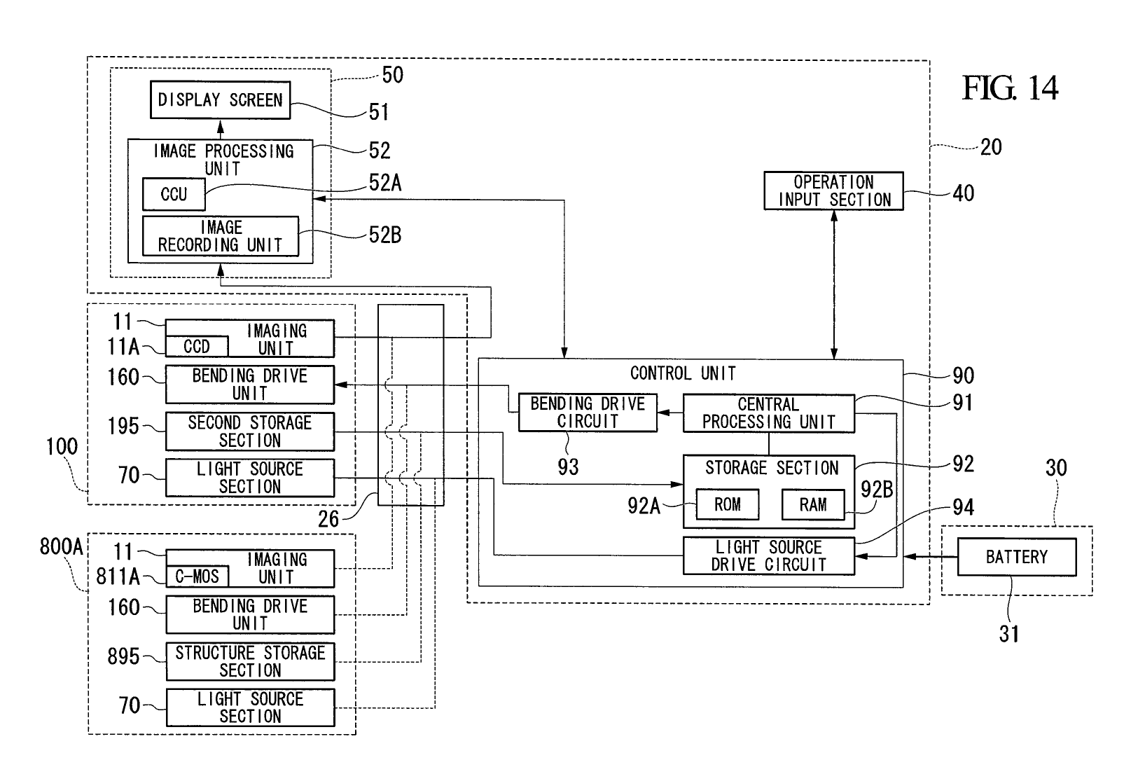

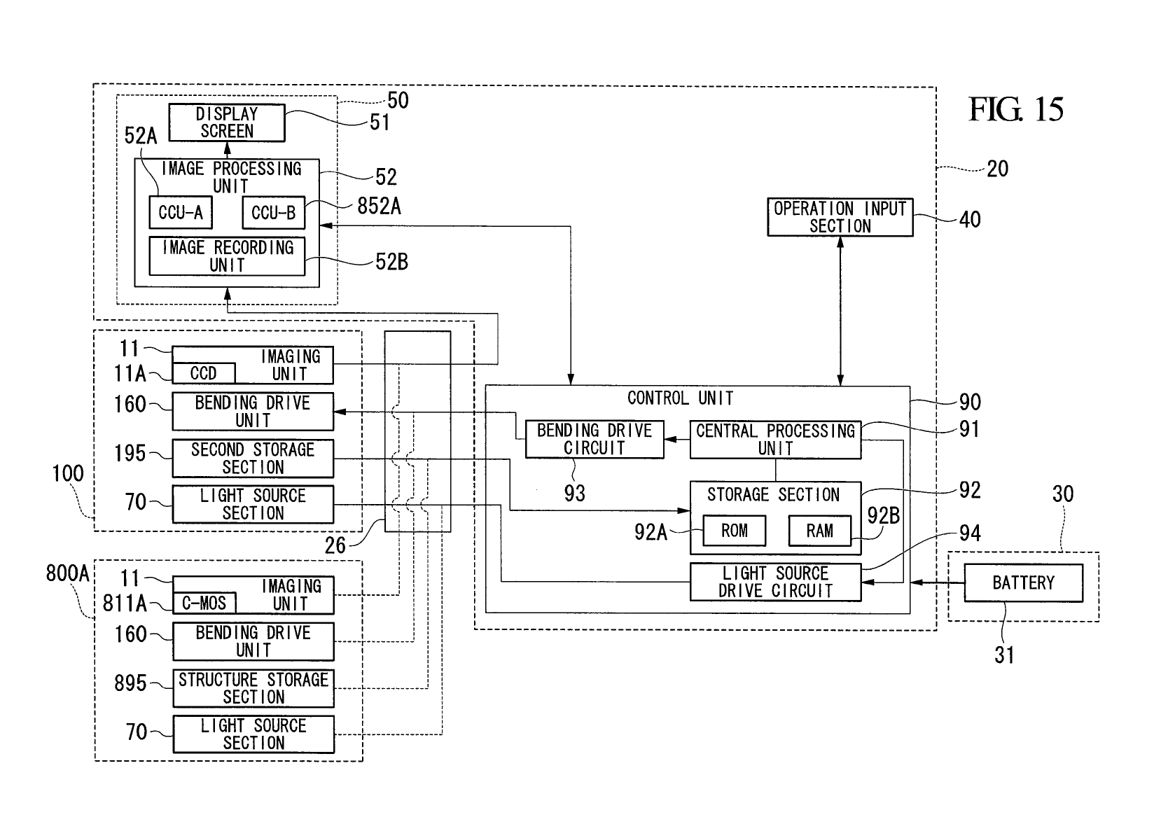

Hereinafter, the structure of variant example 5 of the endoscope system 1 of the present embodiment will be described with reference made to FIG. 14 and FIG. 15. FIG. 14 is a block diagram showing the structure of variant example 5 of the endoscope system 1 of the present embodiment. Moreover, FIG. 15 is a block diagram showing another example of the structure of variant example 5 shown in FIG. 14.

In the scope unit 100 shown in FIG. 14, a CCD 11A is provided in the imaging unit 11, while in the scope unit 800 also shown in FIG. 14, a CMOS 811A is provided in the imaging unit 11 instead of the CCD 11A. The scope unit 800 is a second scope unit which is provided in addition to the scope unit 100.

Moreover, instead of the structure storage section 195, a structure storage section 895 is provided in the scope unit 800. Structure identification information is stored in the structure identification unit 895. The structure identification information includes an operating procedure that is used in processing to convert image information transmitted to the display control unit 52 of the display unit 50 from the CCD 11A or CMOS 811A into signals to be displayed on the display screen 51.

Moreover, structure identification information stored in the structure storage section 195 or the structure storage section 895 is read to the RAM 92B of the control unit 90. For example, when the scope unit 800 is mounted on the main body unit 20, in addition to the parameters used to operate the bending drive unit 160, parameters that are used to operate the display control unit 52 of the display unit 50 are also stored in the RAM 92B.

The display control unit 52 converts image signals transmitted to the display control unit 52 from the CMOS 811A into image signals to be displayed on the display screen 51 based on the parameters stored in the RAM 92B.

In the present variant example, even though different imaging elements which are provided in the imaging unit 11 is different, by reading the structure identification information from the scope unit 100 or scope unit 800 to the RAM 92B of the control unit 90, it is possible to convert the image signals from the imaging element into a format that enables the images to be displayed on the display screen 51. As a result of this, the present variant example makes it possible to switch a plurality of scope units having different types of imaging element and to use these scope units.

Note that operations of the display control unit 52 which are based on parameters are not limited to being a switching structure, and it is also possible to provide a plurality of (for example, the CCU 52A and the CCU 852A shown in FIG. 15) different camera control units on the display control unit 52, and to select the camera control unit to be used based on the structure identification information.

Moreover, as another example of a scope unit having a different type of imaging element, it is possible, for example, to employ a scope unit that employs an imaging element having a different effective pixel number from that of the CCD 11A. If, for example, a CCD is employed that has a greater effective pixel number than that of the CCD 11A, then it is possible to obtain a superior enlarged image even if the maximum zoom magnification obtained from digital zoom processing thereof is greater than in the scope unit 100 in which the CCD 11A is mounted. In accordance with this, it is possible for the structure identification information to include information about the maximum magnification of the digital zoom of the display unit 50. In this case, in imaging elements that have a smaller effective pixel number, the zoom is not increased to a magnification that prevents a specimen object being properly observed, while in imaging elements that have a greater effective pixel number, high-magnification observation is possible without any special settings needing to be made, and the endoscope system 1 can be operated easily.

### Variant Example 6

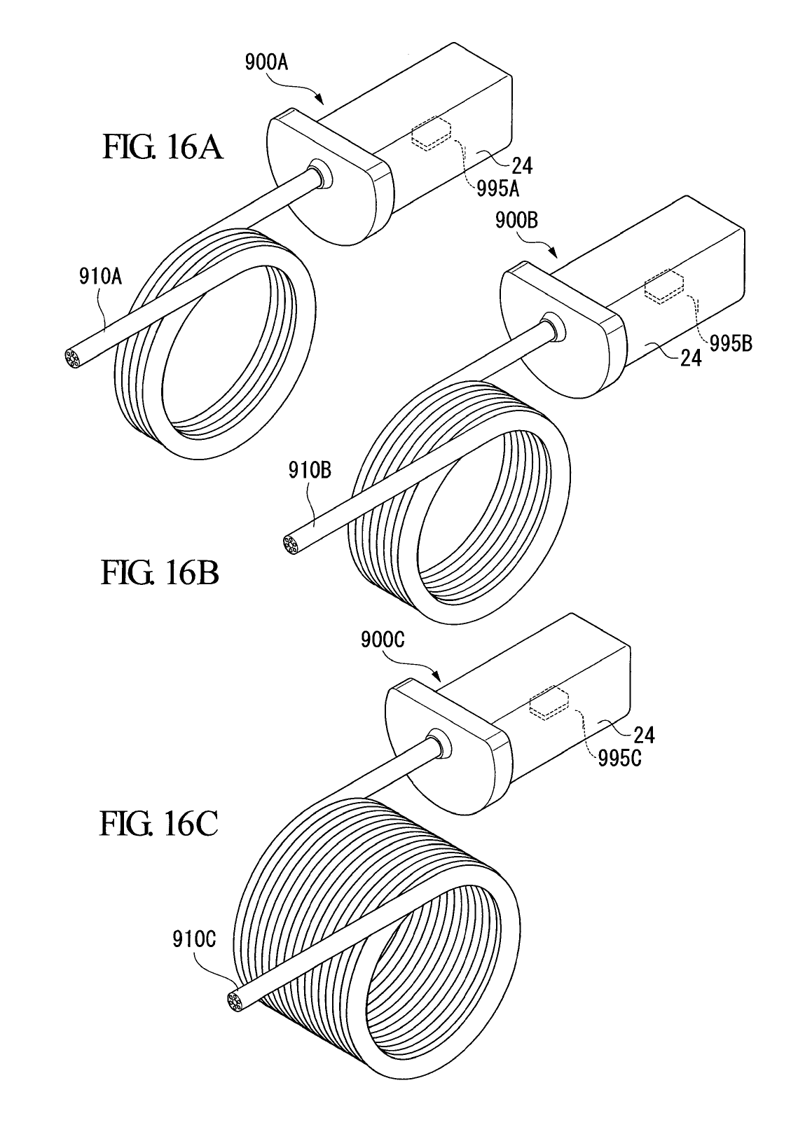

Hereinafter, the structure of variant example 6 of the endoscope system 1 of the present embodiment will be described with reference made to FIG. 16A through FIG. 16C. FIG. 16A is a block diagram showing a scope unit that is able to be mounted on the endoscope system 1 of the present embodiment. FIG. 16B is a block diagram showing another scope unit that is able to be mounted on the endoscope system 1 of the present embodiment. FIG. 16C is a block diagram showing yet another scope unit that is able to be mounted on the endoscope system 1 of the present embodiment.

A scope unit 900A, a scope unit 900B, and a scope unit 900C that are shown in FIG. 16A through FIG. 16C are second scope units that can be provided in addition to the scope unit 100 and scope unit 200.

The scope unit 900A, the scope unit 900B, and the scope unit 900C are provided respectively with a second insertion portion 910A, a second insertion portion 910B, and a second insertion portion 910C in which the same LED 771 as in the scope unit 700 is provided. The second insertion portion 910A, the second insertion portion 910B, and the second insertion portion 910C have mutually different lengths in the axial direction. For example, the length of the second insertion portion 910A is set to two meters, the length of the second insertion portion 110B is set to 10 meters, and the length of the second insertion portion 110C is set to 50 meters. Moreover, CCD 11A are provided respectively at distal ends of the second insertion portion 910A, the second insertion portion 910B, and the second insertion portion 910C.

Moreover, a structure storage section 995A, a structure storage section 995B, and a structure storage section 995C in which structure identification information that is set in accordance with each one of the second insertion portion 910A, the second institution portion 910B, and the second insertion portion 910C is stored respectively are provided inside the unit main body 24. The structure identification information that is stored in the structure storage section 995A, the structure storage section 995 B, and the structure storage section 995C includes parameters that are used to correct image signals transmitted from the CCD 11A of the imaging unit 11 and received by the display control unit 52.

In the present variant example, the same imaging element in the form of the CCD 11A is provided in each one of the second insertion portion910A, the second institution portion 910B, and the second insertion portion 910C. However, image signals transmitted from the CCD 11A to the display control unit 52 have sometimes different electrical characteristics when they arrive at the display control unit 52 in accordance with the length of the insertion portion.

Here, the structure identification information which is stored in each one of the second insertion portion 910A, the second insertion portion 910B, and the second insertion portion 910C is read to the RAM 92B of the control unit 90. Based on the structure identification information read to the RAM 92B, the image signals that arrive at the display control unit 52 are corrected and converted into video signals capable of being displayed on the display screen 51.

In the present variant example, even if scope units having insertion portions of different lengths are switched and used, it is possible to prevent the video displayed on the display screen 51 being corrupted.

Embodiments of the present invention have been described in detail above with reference made to the drawings, however, the specific structure of the present invention is not limited to these embodiments and various design modifications and the like are included therein insofar as they do not depart from the argument of the present invention.

For example, in the above described embodiment, examples are given in which the number of motors 61 is different in the scope unit 100 and the scope unit 200. However, the present invention is not limited to this structure, and it is also possible to switch between a plurality of scope units whose structures differ in that the size or output of the motors is different, and to use these respective scope units. In these cases as well, by providing structure identification information in the scope unit, an operation input in the main body unit can be accurately reflected to the scope unit.

Moreover, in the above described embodiment, examples are given in which the structure identification information contains parameters that serve as the information identifying the structure such as the bending portion, illumination unit, and imaging unit and the like of a scope unit. However, the present invention is not limited to this structure and as the structure identification information it is also possible to store a drive program that is used to drive the bending portion, illumination unit, and imaging unit and the like of a scope unit in the structure storage sections of all of the scope units. In this case, the amount of information being stored in the main body unit can be reduced. Furthermore, it is also possible to transmit a drive program that has not been set in the main body unit from the scope unit side to the main body unit, and generate drive signals that correspond to that scope unit in the main body unit.

Moreover, in the above described embodiment, it is also possible to employ a structure in which an operating system that has been stored in the ROM of the main body unit is stored in the structure storage section of a scope unit, and a simple type of operating system having a function of displaying on a display screen, for example, images stored in the display unit is loaded in the ROM of the main body unit. In this case, operating systems that perform operations appropriate to a scope unit are switched for each individual scope unit and then used.

Moreover, in the above described embodiment, examples are given in which the structure identification information is information that identifies the physical structure of a scope unit. However, the present invention is not limited to this structure and a structure may also be employed in which the structure identification information contains information relating to the operating mode or electrical characteristics of the scope units. For example, it is also possible to identify scope units that, in spite of having the same physical structure, have different operating procedures based on the structure identification information, and to generate drive signals for these scope units in the control unit.

Moreover, component elements illustrated in the above described embodiment and variant examples can also be combined together to form suitable structures. For example, in the same way as a list of light emission methods of the light source section of the scope unit is displayed on the display screen of the display unit in the above described variant example 4, it is possible to include information that is used to display on the display screen a list that is used to select a specific operating method for each scope unit in the structure identification information.

Moreover, as well as being provided in addition to the scope units 100 and 200, the scope units described in each of the above described variant examples may also be provided, for example, as replacements for the scope units 100 and 200.

According to the endoscope device of the present invention, because structure identification information is stored in a storage section provided in a scope unit, it is possible to identify the structure of a scope unit by referring to the structure identification information in a main body unit. As a result of this, operational input into the main body unit can be accurately reflected to the scope unit.

While preferred embodiments of the invention have been described and illustrated above, it should be understood that these are exemplary of the invention and are not to be considered as limiting. Additions, omissions, substitutions, and other modifications can be made without departing from the argument of the present invention. Accordingly, the invention is not to be considered as limited by the foregoing description and is only limited by the scope of the appended claims.

1. An endoscope device having:
a first scope unit that is used to observe a specimen subject; and
a main body unit that can be removably attached to the first scope unit, and, when the first scope unit is loaded in position, controls the first scope unit by communicating with the first scope unit, wherein
the first scope unit has: an insertion portion; a bending portion that is provided on the insertion portion; a bending drive unit that drives the bending portion; an observation device that is provided on the insertion portion and acquires images of the specimen subject; and a storage section in which structure identification information that is based on the structure of the first scope unit is stored, and
the main body unit has: a display unit that displays images acquired by the observation device; an operating section that is used to make operation inputs to operate at least the bending portion; and a control unit that controls the bending drive unit based on the structure identification information stored in the storage section.

2. The endoscope device according to claim 1, wherein the operating section has an operation input section that makes operation inputs into at least one of the bending portion and the display unit, and,
based on the structure identification information, the control unit separates the operation inputs that are input into the operation input section into drive operation inputs that are used to drive the bending drive unit and command operation inputs that specify coordinates on the display unit, and then recognizes these operation inputs.

3. The endoscope device according to claim 2, wherein there is further provided a second scope unit that can be removably attached to the main body unit, and
the second scope unit has: an insertion portion; an observation device that is provided on the insertion portion and acquires images of the specimen subject; and a storage section in which structure identification information that is based on the structure of the second scope unit is stored, and,
when the second scope unit is loaded in main body unit, based on the structure identification information for the second scope unit, the control unit recognizes operation inputs that are input into the operation input section as command operation inputs.

4. The endoscope device according to claim 1, wherein the structure identification information contains parameters that are used to make the bending portion perform a bending operation.

5. The endoscope device according to claim 4, wherein the parameters contain information identifying directions in which the bending portion is able to perform a bending operation.

6. The endoscope device according to claim 4, wherein the parameters contain information that is used to drive the bending drive unit and that is set based on at least one of the length and the diameter of the insertion portion.

7. The endoscope device according to claim 5, wherein the parameters contain information that is used to drive the bending drive unit and that is set based on at least one of the length and the diameter of the insertion portion.
